(19) United States
(12) Patent Application Publication
(10) Pub. No.: US 2013/0323303 A1
(43) Pub. Date: Dec. 5, 2013

(54) DRUG CARRIER AND PREPARATION METHOD THEREOF

(71) Applicants: San-Yuan CHEN, Hsinchu City (TW); Chia-Wei SU, Taoyuan County (TW); Dean-Mo LIU, Hsinchu County (TW)

(72) Inventors: San-Yuan CHEN, Hsinchu City (TW); Chia-Wei SU, Taoyuan County (TW); Dean-Mo LIU, Hsinchu County (TW)

(73) Assignee: NATIONAL CHIAO TUNG UNIVERSITY, Hsinchu City (TW)

(21) Appl. No.: 13/607,829

(22) Filed: Sep. 10, 2012

(30) Foreign Application Priority Data
May 29, 2012 (TW) 101119161

(51) Int. Cl. A61K 9/48; A61K 31/704; B82Y 5/00

(52) U.S. Cl. 424/455; 514/34; 977/798; 977/906

(57) ABSTRACT

A drug carrier is provided with a structure of a lipid shell enclosing aqueous micelles. The lipid shell includes lipid and emulsifier, in which the emulsifier encloses the lipid. The components of the aqueous micelles are phospholipids and amphiphilic chitosan, and the aqueous micelles enclose an aqueous solution containing a drug. Furthermore, the method of preparing the drug carrier is also provided. Therefore, with the pharmaceutical advantages of lipid-based nanoparticle included low drug leakage and the ability of to overcome the multiple drug resistance, this new formulation were further incorporated with the chitosan and featured with high payload efficiency. The features could enhance intracellular concentration of anti-cancer drug and oral bioavailability.

### RELATED APPLICATIONS

This application claims priority to Taiwan Application Serial Number 101119161, filed May 29, 2012, which is herein incorporated by reference.

### BACKGROUND

1. Technical Field Disclosure

The present disclosure relates to a drug carrier and its preparing method. More particularly, the present disclosure relates to an oral drug carrier and its preparing method.

2. Description of Related Art

Since liposome first described in 1965, the liposome has been considered as an ideal dosage form for drug delivery. The liposome can carry anticancer drugs and release them into a tumor region, reducing the possibility of the drugs entering and thus damaging normal cells. However, in the drug clinical trials of liposome, there still exist many problems including lower drug encapsulation rate, high preparation costs, long-term instability, hardly controlled process and minor biological incompatibility.

In addition, though polymer materials can flexibly manipulate the characteristic as polymer carrier by modification, they also have disadvantage of being susceptible to the surrounding temperature and pH value. In the mean time, most of the polymer still has insufficient biological incompatibility, which limits the development of such polymer carrier.

In the study of malignant tumor treatments, cancer cells exhibit multiple drug resistance, so that the traditional anti-cancer drugs cannot be accumulated to a sufficient amount in the cells, thus limiting the therapeutic efficiency of the drugs. Multiple drug resistance is attributed to the overexpression of P-glycoprotein (P-gp) in cells of the normal tissue (such as small intestine cells), and many anticancer drugs are substrates of P-gp, which significantly deteriorate their oral bioavailability and eliminate the oral administration possibility.

Given the above, in the drug administration of disease treatments, particularly the treatment of malignant cancers, there still needs an oral drug carrier with enhanced stability to enhance the dosing effect and applicability.

### SUMMARY

The present disclosure combines both features of a polymer micelle and a lipid particle to prepare an oral drug carrier having high encapsulation rate and low rate of drug leakage, and capable of overcome the multiple drug resistance.

An aspect of the present disclosure is to provide an oral drug carrier, composed of a lipid shell enclosing a plurality of aqueous micelles, and the aqueous micelles are dispersed uniformly within the lipid shell. The lipid shell comprises a lipid and an emulsifier, and the emulsifier encloses the lipid; the aqueous micelles comprise a phospholipid and a chitosan, and the aqueous micelles enclose an aqueous solution containing a drug.

According to an embodiment of the present disclosure, the above-mentioned emulsifier is sodium cholate, sodium glycocholate, sodium taurocholate, sodium taurodeoxycholate, poloxamer, tween, polyvinyl alcohol or ethoxylated hydrogenated castor oil. The lipid is glycerol tripalmitate, Dynasan 112, Dynasan 114, Dynasan 118, monostearin, distearin, tristearin, stearic acid, palmitic acid or cholesterol.

According to another embodiment of the present disclosure, the chitosan is an amphiphilic chitosan. The phospholipid is lecithin, soybean lecithin, egg yolk lecithin or a synthetic phospholipid.

According to another embodiment of the present disclosure, the drug is doxorubicin.

According to yet another embodiment of the present disclosure, the diameter of the oral drug carrier is in the range of about 100 nm to about 500 nm.

Another aspect of the present disclosure is to provide a method of preparing an oral drug carrier with drug resistance. The first step is to prepare a first aqueous solution and an organic solution, the first aqueous solution contains a chitosan and an aqueous solution containing a drug, and the organic solution contains a lipid, a phospholipid and an organic solvent. The next step is to mix the first aqueous solution and the organic solution, the chitosan and the phospholipid self-assemble to form an aqueous micelle or a plurality of aqueous micelles, and the aqueous micelles are dispersed in the lipid to form a first emulsion of a water-in-oil type. Then the first emulsion s added to a second aqueous solution, and the first emulsion is dispersed uniformly in the second aqueous solution to form a second emulsion of a water-in-oil-in-wager type after sonication. And the organic solvent of the second emulsion is removed to obtain a plurality of oral drug carriers dispersed uniformly in the second aqueous solution.

According to an embodiment of the present disclosure, the drug is doxorubicin.

According to an embodiment of the present disclosure, the second aqueous solution contains a sodium cholate as an emulsifier, and the concentration of the sodium cholate is about 1% w/v.

According to another embodiment of the present disclosure, the concentration of the chitosan in the first aqueous solution is about 0,01% w/v to about 5% w/v, preferably about 0.05% w/v to about 2% w/v.

According to another embodiment of the present disclosure, the lipid is glycerol tripalmitate, and the concentration of glycerol tripalmitate is about 0.2% w/v to about 0.5% w/v, The phospholipid is lecithin, and the concentration is about 0.15% w/v to about 0.4% w/v. The organic solvent is chloroform.

According to yet another embodiment of the present disclosure, the method for mixing is using an ultrasonic processor.

According to yet another embodiment of the present disclosure, further comprising a step of removing water from the second aqueous solution containing the oral drug carriers to obtain the oral drug carrier in powder form after the step of removing the organic solvent.

It is to be understood that both the foregoing general description and the following detailed description are by examples, and are intended to provide further explanation of the disclosure as claimed.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The disclosure can be more fully understood by reading the following detailed description of the embodiment, with reference made to the accompanying drawings as follows:

FIG. 1A and 1B is a schematic diagram of an oral drug carrier according to one embodiment of this disclosure;

FIG. 2 is a flow diagram of a method for preparing an oral drug carrier according to one embodiment of this disclosure;

FIG. 3A is a transmission electron microscopic image of an oral drug carrier according to one embodiment of this disclosure;

FIG. 3B is a transmission electron microscopic image of an aqueous micelle in an oral drug carrier according to one embodiment of this disclosure;

FIG. 4 is a drug release rate graph of an oral drug carrier at different pH environments according to one embodiment of this disclosure;

FIG. 5A is a confocal microscopic image in a permeability test of an oral drug carrier across caco-2 cell monolayers according to one embodiment of this disclosure;

FIG. 5B is a confocal microscopic image in permeability testing of an oral drug carrier across the caco-2 cell monolayers according to one embodiment of this disclosure;

FIG. 6A is an IVIS picture of a mouse model treated with drugs at 0 day;

FIG. 6B is an IVIS picture of a mouse model treated with drugs after 28 days;

FIG. 6C is an IVIS picture of a mouse model treated with drugs at 0 day;

FIG. 6D is an IVIS picture of a mouse model treated with drugs after 28 days; and

FIG. 7 is a variation graph of tumor cells illustrated by a mouse model treated with drugs.

### DETAILED DESCRIPTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

As used herein, the singular forms “a” “an” and “the” include plural referents unless the context clearly dictates otherwise. Therefore, reference to, for example, a micelle includes aspects having two or more such micelles, unless the context clearly indicates otherwise.

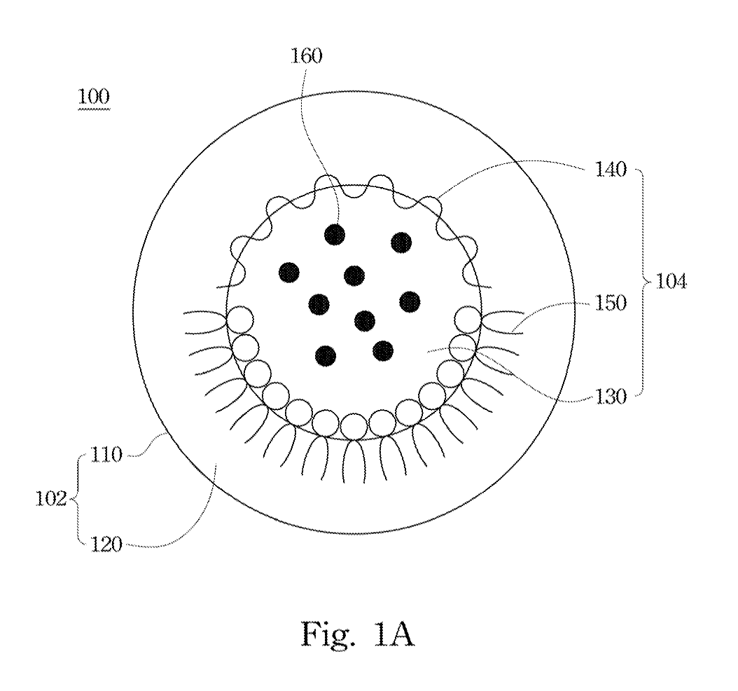

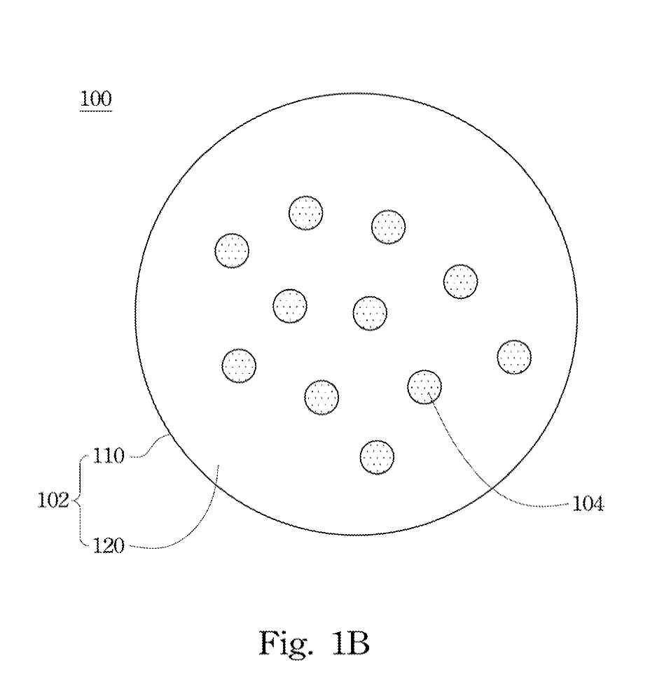

FIG. 1A and FIG. 1B illustrate a schematic diagram of an oral drug carrier 100 according to the present disclosure. The oral drug carrier 100 is composed of a plurality of aqueous micelles 104 dispersed uniformly in a lipid shell 102. FIG. 1A illustrates an aqueous micelle 104 to describe the structure of the oral drug carrier 100 more clearly. As shown in FIG. 1A the lipid shell 102 comprises an emulsifier 110 and a lipid 120, and the emulsifier 110 encloses the lipid 120. The aqueous micelle 104 encloses an aqueous solution 130 containing a drug 160. The aqueous micelle 104 comprises a chitosan 140 and a phospholipid 150. FIG. 1B shows an oral drug carrier 100 comprising a plurality of aqueous micelles 104 dispersed in a lipid shell 102.

The emulsifier 110 of the lipid shell 102 is contributive to disperse the hydrophobic molecules in the solution. According to an embodiment, the emulsifier 110 is sodium cholate, sodium glycocholate, sodium taurocholate, sodium taurodeoxycholate, poloxamer, tween, polyvinyl alcohol or ethoxylated hydrogenated castor oil.

The lipid 120 in the lipid shell 102 is a solid lipid having high stability to the environmental pH value and temperature. According to an embodiment, the lipid 120 is glycerol tripalmitate Dynasan 112, Dynasan 114, Dynasan 118, monostearin, distearin, tristearin, stearic add, palmitic acid or cholesterol.

The chitosan modified by hydrophobic hexanoyl and hydrophilic carboxymethyl acid is an amphiphilic chitosan, so the chitosan has the hydrophilic and the hydrophobic properties simultaneously. This kind amphiphilic micromolecule is dissolved in water for forming micelles.

According to an embodiment, the drug 160 is doxorubicin,

The above oral drug carrier 100 is a core-shell nano-structure particle, and the diameter of the oral drug carrier is in the range of about 100 nm to about 500 nm, preferably about 110 nm to about 200 nm, more preferably about 120 nm to about 150 nm.

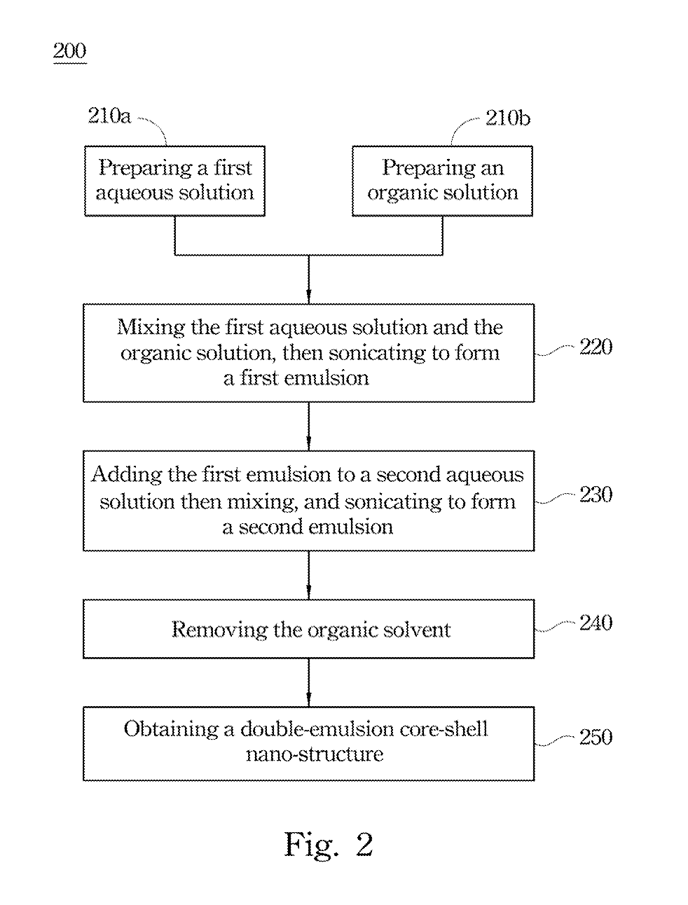

FIG. 2 illustrates a flow diagram of a method for preparing an oral drug carrier. The preparing method 200 as shown in FIG. 2, the first step is to prepare a first aqueous solution 210a and an organic solution 210b, and the two solutions are stirred and mixed to form a first emulsion of a water-in-oil type 220. Then the first emulsion is added to a second aqueous solution for forming a second emulsion of a water-in-oil-in-water type 230 after stirring and mixing. Later an organic solvent 240 of the second emulsion is removed to obtain a plurality of oral drug carriers 250 dispersed uniformly in the second aqueous solution. In step 210a, the first aqueous solution contains a chitosan and a drug, and the concentration of the chitosan is about 0.01% w/v to about 5% w/v, preferably about 0.05% w/v to about 2% w/v. In an embodiment, the drug is doxorubicin,

In step 210b, a lipid and a phospholipid are dissolved in an organic solvent for forming the organic solution. In an embodiment, the lipid is glycerol tripalmitate, and the concentration is about 0.2% w/v to about 0.5% w/v. The phospholipid is lecithin, and the concentration is about 0.15% w/v to about 0.4% w/v. The organic solvent is chloroform.

In step 220, the first aqueous solution and the organic solution are mixed, so the chitosan and the phospholipid self-assemble to form an aqueous micelle or a plurality of aqueous micelles dispersed in the lipid for forming the first emulsion of a water-in-oil type. The drug is enclosed within the aqueous micelles.

In step 230, the first emulsion is added to the second aqueous solution, and the first emulsion is dispersed uniformly in the second aqueous solution to form the second emulsion of a water-in-oil-in water type. The above second emulsion contains an emulsifier. In an embodiment, the emulsifier is a sodium cholate aqueous solution, and the concentration of the sodium cholate aqueous solution is preferably about 1% w/v.

A mixing method in the above step 220 and step 230 is using an ultrasonic processor.

In step 240, the organic solvent within the second emulsion is removed to obtain a plurality of oral drug carriers dispersed uniformly in the second aqueous solution. In an embodiment, the method of removing the organic solvent is using a rotary vacuum evaporator.

After step 240, further comprising a step of removing water from he second emulsion to obtain an oral drug carrier in powder formulations by freeze-drying method. A solution having an oral drug carrier is dispensed to centrifuge tubes and placed them in freeze-drying bottles. Adding appropriate amount of liquid nitrogen to the freeze-drying bottles making the solution freeze into a solid. Then the freeze-drying bottles is connected to a freeze dryer in an environment of −40° C. and 0.133 mBar for one day, thus obtaining the dry powdered oral drug carrier.

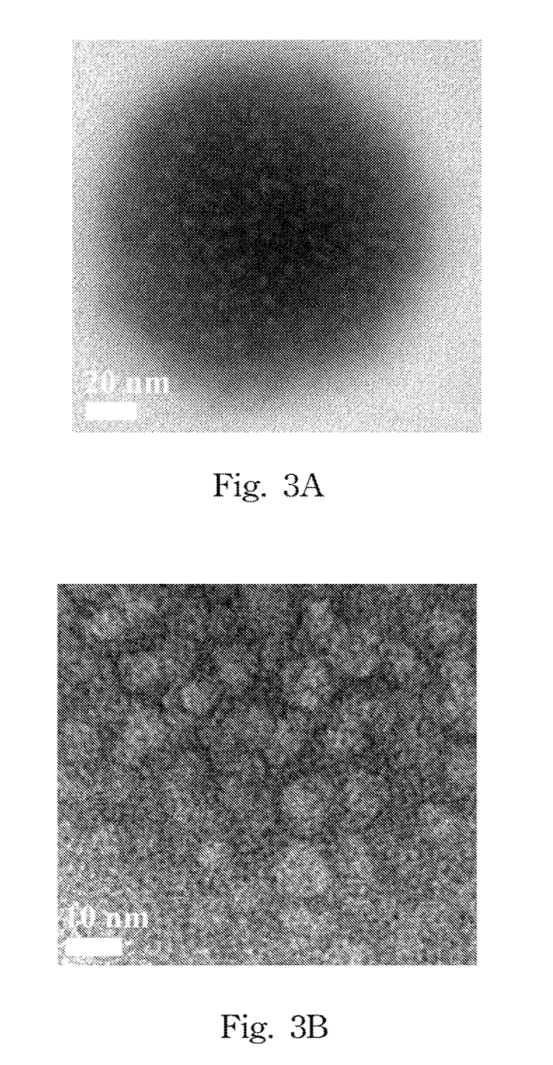

An oral drug carrier manufactured by an embodiment in the present disclosure is shown in FIG. 3A. FIG. 3B shows a magnified portion of the aqueous micelles in FIG. 3A, as shown in the figure, the drug is dispersed uniformly in the aqueous phase micelles.

### EXAMPLE 1

In Example 1 anticancer drug Doxorubucin was used as the enclosed drug. Referring to the flow diagram of FIG. 2 for preparing an oral drug carrier and the description of the above embodiments, 1 mg doxorubicin hydrochloride first dissolved in deionized water, and the appropriate amount of a water-soluble chitosan modified by carboxymethyl groups was added the above aqueous solution for forming a first aqueous solution at the concentration of 0.05% w/v. Then glycerol tripalmitate and lecithin were dissolved in 1 mL chloroform for forming an organic solution at the concentration of 0.5% w/v and 0.15% w/v. After the first aqueous solution containing doxorubucin adding to the organic solution, the above organic solution was mixed and emulsified by an ultrasonic processor for forming a first emulsion of a water-in-oil type.

The first emulsion was added to a second aqueous solution containing 1% w/v sodium cholate, and then mixed by the ultrasonic processor for forming a second emulsion of a water-in-oil-in-water type. After removing the chloroform by rotary vacuum evaporator, an oral drug carrier was precipitated and dispersed stably in the solution.

### EXAMPLE 2

In Example 2, anticancer drug doxorubucin was used as the enclosed drug. Referring to the flow diagram of FIG. 2 for preparing an oral drug carrier and the description of the above embodiments, 1 mg doxorubicin hydrochloride was first dissolved in deionized water, and the appropriate amount of a water-soluble chitosan modified by carboxymethyl groups was added the above aqueous solution for forming a first aqueous solution at the concentration of 0.05% w/v. Afterwards, glycerol tripalmitate and lecithin were dissolved in 1 mL chloroform for forming an organic solution at the concentration of 0.2% w/v and 0.4% w/v. After the first aqueous solution containing doxorubucin being added to the organic solution, the above organic solution was mixed and emulsified by an ultrasonic processor for forming a first emulsion of a water-in-oil type.

The first emulsion was added to a second aqueous solution containing 1% w/v sodium cholate, and mixed by the ultrasonic processor for forming a second emulsion of a water-in-oil-in-water type. After removing the chloroform by rotary vacuum evaporator, an oral drug carrier was precipitated and dispersed stably in the solution.

As shown in FIG. 3A and 3B, an oral drug carrier having a core-shell nano-structure was observed by transmission electron microscopy (TEM). Therefore, changing the ratio of glycerol tripalmitate and lecithin can affect the types of the double emulsion core-shell nano-structure.

### EXAMPLE 3

According to the flow diagram of FIG. 2 and the above embodiments, an oral drug carrier was prepared by chitosan at different concentration referring to table 1, and was analyzed with the related characteristics. As shown in table 1, when the concentration of the chitosan was 0.05%, the efficiency of drug enclosed by the oral drug carrier was higher. Accordingly, lower concentration of the chitosan decreased amount of the enclosed drug. And the overly high concentration of the chitosan decreases the solubility of drug, so as not to enclose more amounts of drugs.

TABLE 1
A characteristic analysis table of the oral drug carrier prepared
by chitosan at different concentration.

| | | Average | | |
|---|---|---|---|---|
| | Concentration | particle size | Surface | Encapsulation |
| Sample | of chitosan (%) | (nm) | potential (mV) | efficiency (%) |
| 1 | 0.01 | 179.5 ± 3.2 | −29.21 ± 0.56 | 68.25 ± 1.75 |
| 2 | 0.05 | 181.3 ± 2.1 | −30.70 ± 0.48 | 78.95 ± 2.71 |
| 3 | 0.2 | 183.5 ± 3.6 | −31.54 ± 1.02 | 76.35 ± 3.12 |
| 4 | 1 | 190.4 ± 5.5 | −32.82 ± 0.98 | 71.24 ± 1.89 |
| 5 | 3 | 205.6 ± 7.5 | −29.35 ± 1.83 | 69.53 ± 2.52 |
| 6 | 5 | 217.4 ± 5.8 | −27.28 ± 0.78 | 65.31 ± 3.29 |

* Encapsulation efficiency (%) = Doxo loading capacity/Doxo total amount × 100%

As such, the concentration of the chitosan can affect the encapsulation efficiency of drug and the particle size of the oral drug carrier.

### EXAMPLE 4

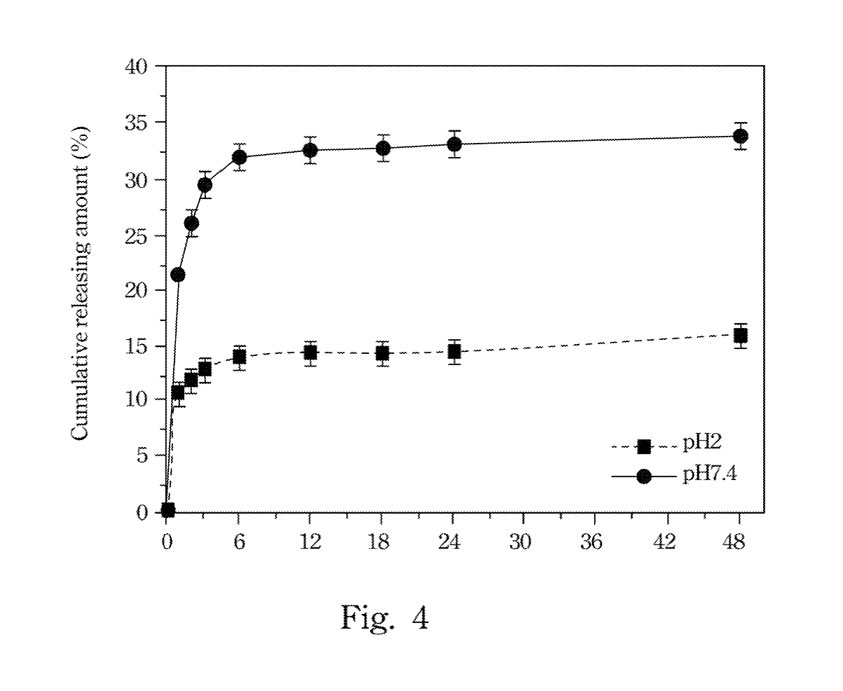

An oral drug carrier was prepared according to the flow diagram of FIG. 2 and the above embodiments, and anticancer drug doxorubucin was used as the enclosed drug. The drug release rate was evaluated at environments of different pH values. As shown in FIG. 4, the drug cumulative releasing amount in an environment of pH 2 was lower than the drug cumulative releasing amount in an environment of pH 4.

Therefore, the oral drug carrier was affected by the protonation of the amino group of the chitosan and the carboxyl group of the sodium cholate, so the drug release rate was significantly lower in the acidic pH environment than in the neutral environment. The feature, which oral drug carrier can pass through the low pH environment in this way of drug administration, not only protects the enclosed drug, but also decreases the drug leakage.

### EXAMPLE 5

An oral drug carrier was prepared according to the flow diagram of FIG. 2 and the above embodiments. Anticancer drug doxorubucin was used to be the enclosed drug. The intestinal permeability of the oral drug carrier was tested in vitro.

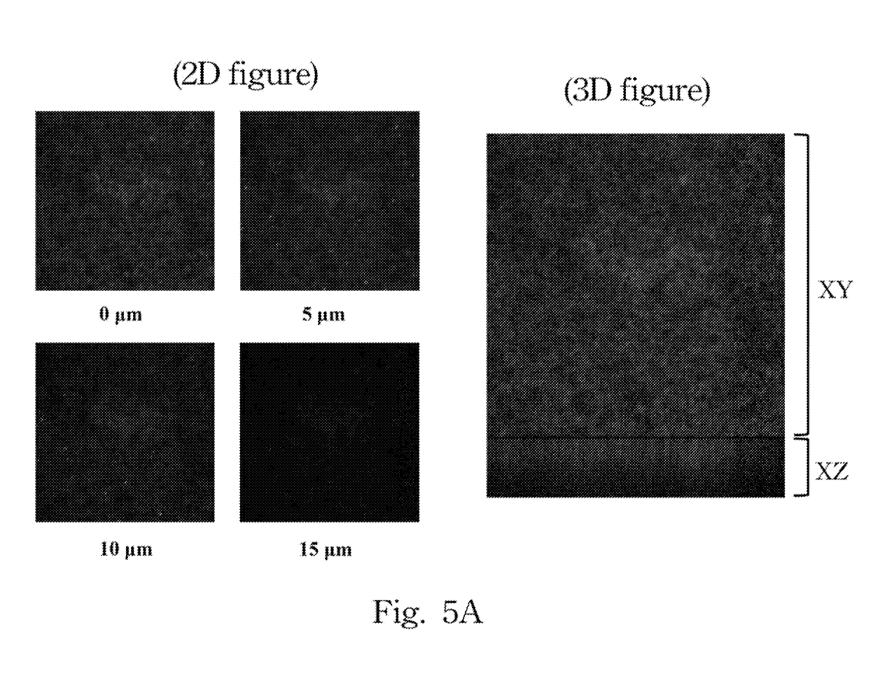

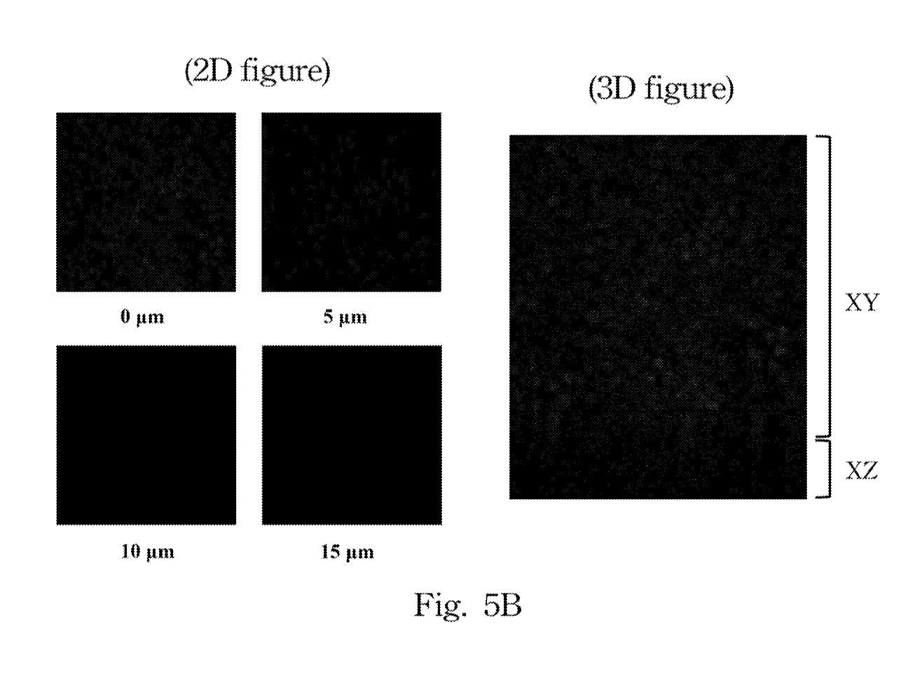

In vitro experiment, caco-2 cell monolayers are often used to evaluate intestinal permeability. FIG. 5A shows confocal microscopic images in permeability testing of the oral drug carrier containing DOXO through caco-2 cell monolayers; FIG. 5B shows confocal microscopic images in permeability testing of DOXO only through the caco-2 cell monolayers. As shown in FIG. 5A, the confocal microscopic images of the oral drug carrier enclosing drug shows visible red fluorescent signals even at 15 μm depth (the red fluorescent signals comes from doxorubucin). However in FIG. 5B, the carrier without doxorubucin only shows the red fluorescent signals on the top layer.

From the above in vitro experiment, the oral drug carrier disclosed in the present disclosure has the effect increasing the intestinal permeability of doxorubucin.

### EXAMPLE 6

An oral drug carrier was prepared according to the flow diagram of FIG. 2 and the above embodiments. Anticancer drug doxorubucin was used as the enclosed drug. The intestinal permeability of the oral drug carrier was tested in vivo.

Under the in vivo experiment of animal tumor model, first a mouse model treated with doxorubucin was prepared as the control group, and another mouse model treated with the oral drug carrier containing doxorubucin was prepared as the experimental group. After drug treatment, the mouse models were recorded the variation of tumor size via in vivo imaging system (IVIS) (because of the mice transplanted with the cancer cells carrying fluorescent gene).

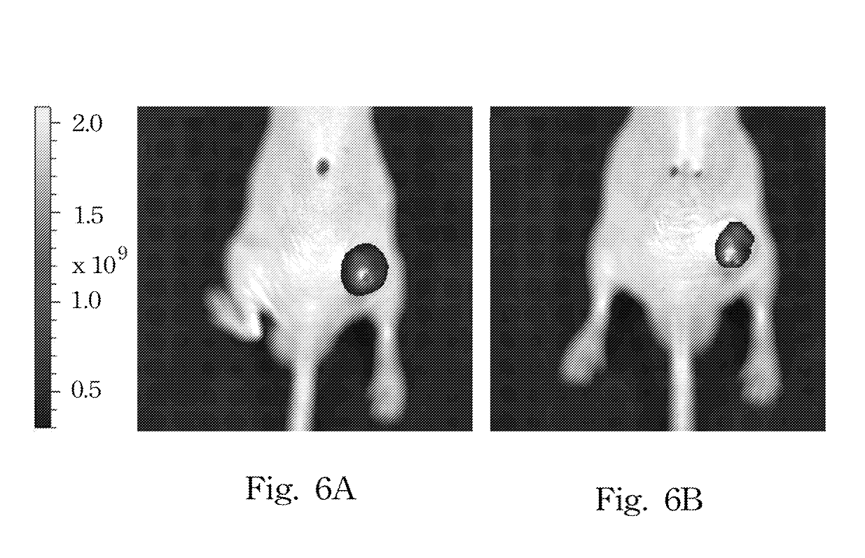

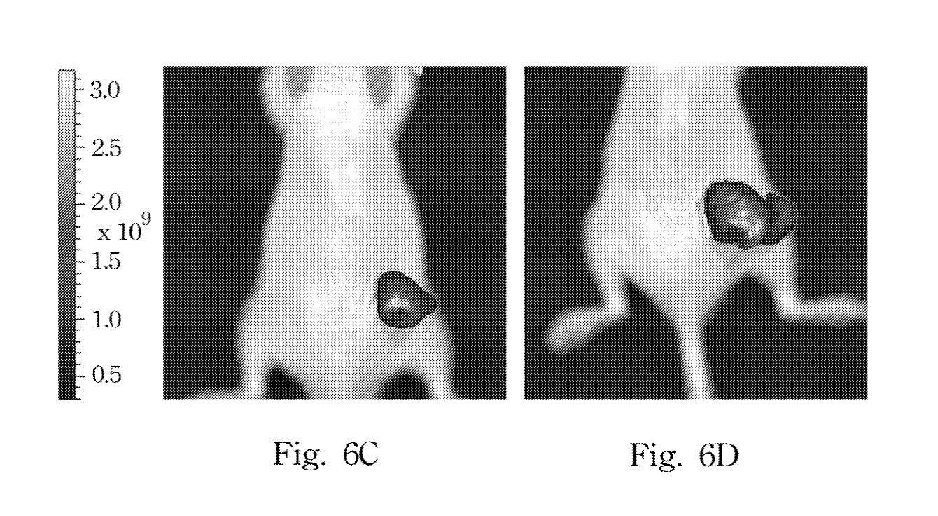

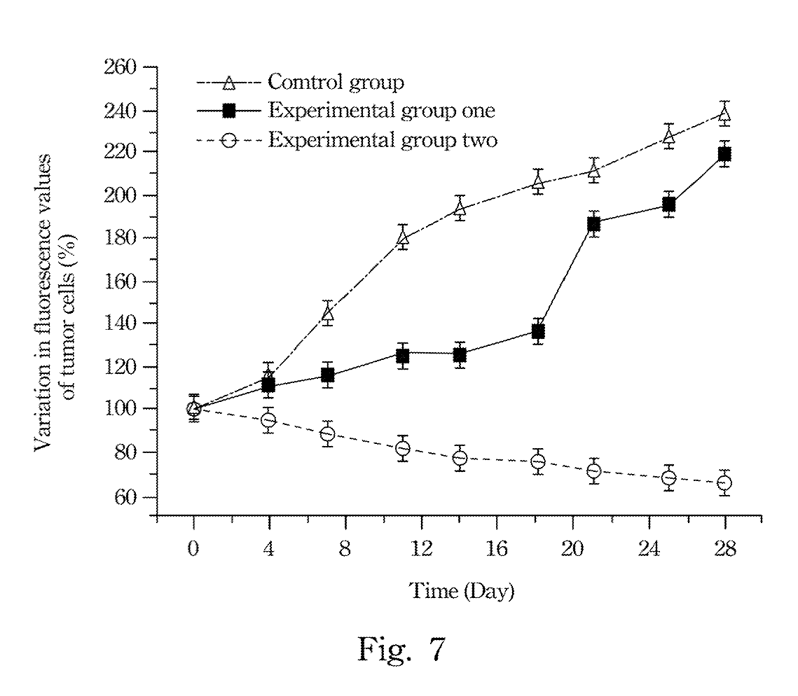

FIG. 6A and 6B are MS pictures of the experimental group that the mouse model was treated with drug at 0 day and after 28 days. The tumor size of the mouse model in the experimental group was 65% compared to before treatment. FIG. 6C and 6D are IVIS photos of the control group that the mouse model was treated with drug at 0 day and after 28 days. As shown in FIG. 6D, the tumor size of the mouse model in the control group still grew up to 200% compared to before treatment. FIG. 7 is a variation graph in fluorescence values of tumor cells tested by IVIS, and the tumor cells were from the above mouse model treated with drug.

The above embodiments/examples in the present disclosure use the properties of lipid particles to prepare an oral drug carrier, and the micron-grade and nano-grade core-shell structure can be applied to the oral drug carrier. In the lipid shell, the amphiphilic chitosan and the lecithin self-assemble to form nano-grade micelles. The chitosan has advantage of less expensive price, high biocompatibility and degradability, as well as flexibility in chemically modification. These features make the micelles enclose each kind of drug effectively, help to increase the payload efficiency, and decrease drug leakage.

The solid lipid nanoparticles formed from the lipid have higher stability to pH value and temperature, and it can improve the properties of high drug leakage and instability resulted from the drug only enclosed by high molecular polymer. Otherwise, lipid can also help to overcome multiple drug resistance for increasing the drug concentration within cells and oral bioavailability. Hope the oral drug carrier can replace the injection formulation to become a new application platform of oral drug carrier for cancer therapy in the future.

All the features disclosed in this specification (including any accompanying claims, abstract, and drawings) may be replaced by alternative features serving the same, equivalent or similar purpose, unless expressly stated otherwise. Thus, each feature disclosed is one example only of a generic series of equivalent or similar features.

What is claimed is:

1. An oral drug carrier comprising:
a lipid shell comprising a lipid and an emulsifier, wherein the emulsifier encloses the lipid: and
a plurality of aqueous micelles comprising a phospholipid and a chitosan and dispersed uniformly within the lipid shell, wherein the aqueous micelles enclose an aqueous solution containing a drug.

2. The oral drug carrier of claim 1, wherein the emulsifier is sodium cholate, sodium glycocholate, sodium taurocholate, sodium taurodeoxycholate, poloxamer, tween, polyvinyl alcohol or ethoxylated hydrogenated castor oil.

3. The oral drug carrier of claim 1, wherein the lipid is glycerol tripalmitate, Dynasan 112, Dynasan 114, Dynasan 118, monostearin, distearin, tristearin, stearic acid, palmitic acid or cholesterol.

4. The oral drug carrier of claim 1 wherein the chitosan is an amphiphilic chitosan.

5. The oral drug carrier of claim 1, wherein the phospholipid is lecithin, soybean lecithin, egg yolk lecithin or a synthetic phospholipid.

6. The oral drug carrier of claim 1, wherein the drug is doxorubicin.

7. The oral drug carrier of claim 1, wherein the diameter of the oral drug carrier is in the range of about 100 nm to about 500 nm.

8. A method of preparing an oral drug carrier comprising:
preparing a first aqueous solution and a organic solution, wherein the first aqueous solution contains a chitosan and an aqueous solution containing a drug, and the organic solution contains a lipid, a phospholipid and an organic solvent;
mixing the first aqueous solution and the organic solution, wherein the chitosan and the phospholipid self-assemble to form an aqueous micelle or a plurality of aqueous micelles containing the aqueous solution containing the drug, and the aqueous micelles are dispersed in the lipid for forming a first emulsion of a water-in-oil type:
adding the first emulsion to a second aqueous solution, wherein the first emulsion is dispersed uniformly in the second aqueous solution for forming a second emulsion of a water-in-oil-in-water type; and
removing the organic solvent of the second emulsion to obtain a plurality of oral drug carriers dispersed uniformly in the second aqueous solution.

9. The method of claim wherein the drug is doxorubicin.

10. The method of claim 8, wherein the second aqueous solution contains a sodium cholate as an emulsifier, and the concentration of the sodium cholate is about 1% w.v.

11. The method of claim 8, wherein the organic solvent is chloroform.

12. The method of claim 8, wherein the concentration of the chitosan in the first aqueous solution is about 0.01% w/v to about 5% w/v.

13. The method of claim 12, wherein the concentration of the chitosan in the first aqueous solution is about 0.05% w/v to about 2% w/v.

14. The method of claim 8, wherein the lipid is glycerol tripalmitate, and the concentration of glycerol tripalmitate is about 0.2% w/v to about 0.5% w/v.

15. The method of claim 8, wherein the phospholipid is lecithin, and the concentration of lecithin is about 0.15% w/v to about 0.4% w/v.

16. The method of claim 8, wherein the method for mixing is using an ultrasonic processor.

17. The method of claim 8, further comprising a step of removing water from the second aqueous solution containing the oral drug carriers to obtain the oral drug carrier in powder form after the step of removing the organic solvent.
